(19) United States
(12) Patent Application Publication
(10) Pub. No.: US 2016/0022605 A1
(43) Pub. Date: Jan. 28, 2016

(54) METHODS FOR REDUCING MICROSATELLITE INSTABILITY INDUCED BY CHEMOTHERAPY AND METHODS FOR SCREENING ANTIOXIDANTS THAT SUPPRESS DRUG-INDUCED MICROSATELLITE INSTABILITY WHILE ENHANCING THE CYTOTOXICITY OF CHEMOTHERAPEUTIC AGENTS

(71) Applicant: Christina Ling CHANG, Tainan City (TW)

(72) Inventor: Christina Ling CHANG, Tainan City (TW)

(21) Appl. No.: 14/804,360

(22) Filed: Jul. 21, 2015

Related U.S. Application Data
(60) Provisional application No. 62/027,447, filed on Jul. 22, 2014.

(51) Int. Cl. A61K 31/12; G01N 33/50; A61K 31/11

(52) U.S. Cl. CPC A61K 31/12; A61K 31/11; G01N 33/5011; G01N 2500/10

(57) ABSTRACT

A therapeutic approach to prevent drug resistance and chemotherapy-related secondary cancer associated with DNA mismatch repair (MMR) deficiency is disclosed based on screening antioxidants for reducing microsatellite instability (MSI) while enhancing the cytotoxicity of chemotherapeutic agents. The work is based on experiments using antioxidants to target reactive oxygen species generated by oxaliplatin, a commonly used chemotherapeutic agent, and is applicable to other chemotherapeutic agent, and in particular 5-fluorouracil, methotrexate, CCNU, etoposide and vinblastine. In particular oxaliplatin is co-treated with an antioxidant, including CDC, CAPE, ciclopirox ethanolamine, hinokitiol, gossypol, n-Octyl caffeate, baicalein, or curcumin.

### CROSS REFERENCE TO RELATED APPLICATION

This application claims the benefit of filing date of U.S. Provisional Application Ser. No. 62/027,447, entitled “METHOD FOR INHIBITING CHEMOTHERAPY-INDUCED MICROSATELLITE INSTABILITY” filed Jul. 22, 2014 under 35 USC §119(e)(1).

### BACKGROUND OF THE INVENTION

1. Field of the Invention

The present invention relates to materials and methods for screening antioxidants that suppress drug-induced microsatellite instability (MSI) while enhancing the cytotoxicity of chemotherapeutic agents. Further, methods are provided for reducing microsatellite instability induced by chemotherapy while enhancing drug mediated cytotoxicity, which comprises administering a therapeutically effective amount of an antioxidant to an individual receiving the chemotherapy.

2. Description of Related Art

Next-generation sequencing of multiple cancers has revealed that every cancer harbors a large collection of mutations. Furthermore, cancers of different origins display tremendous complexity and heterogeneity in the patterns of mutations, which complicates the design of potential therapeutic approaches that precisely target the underlying molecular pathway(s) of individual cancers. Chemotherapy remains as a mainstream treatment for cancer patients. However, Chemotherapy is closely linked to microsatellite instability (MSI), a hallmark of DNA mismatch repair (MMR) deficiency that is believed to contribute to cancer pathogenesis and drug resistance.

The MMR system also minimizes mutations caused by DNA-damaging agents such as chemotherapeutic agents. The hMutSα complex recognizes a range of DNA lesions [Heinen, 2014; O'Brien and Brown, 2006], and apparently acts as a sensor in the DNA damage response network [Marechal and Zou, 2013]. For example, MMR recognizes the FdU:G mispairing generated by 5-fluorouracil (5-FU) and inter-strand crosslinks generated by CCNU-modified O⁶-(2-chloroethyl) guanine [Meyers et al., 2005; Fischer et al., 2007; Aquilina et al., 1998; Fiumicino et al., 2000]. After exposure to DNA-damaging agents, hMutSα and hMutLα complexes interact with ATM and ATR and initiate MMR-dependent DNA damage response [Stojic et al., 2004; Yoshioka et al., 2008; Kim et al., 2011]. If the DNA repair is successful, cells will exit the checkpoints and resume cell-cycle progression. If DNA repair is unsuccessful, cells will undergo apoptosis [Su, 2006; Duckett et al., 1999]. When the MMR function is deficient, cells develop DNA damage tolerance and become resistant to certain chemotherapeutic agents including 5-FU, methotrexate and cisplatin [Fink et al., 1996; Carethers et al., 1999; Martin et al., 2009; Fink et al., 1997].

In addition to drug resistance, MMR deficiency also contributes to cancer pathogenesis. Germline mutations of MMR genes such as hMSH2, hMSH6, hMLH1 and/or hPMS2 occur in most of patients with Lynch Syndrome [Lynch et al., 2009]. MSI is also detected in ˜15% of sporadic cancers, including colorectal, breast and prostate cancers, due to genetic and/or epigenetic alterations [Peltomaki, 2003; Nowacka-Zawisza et al., 2006; Dahiya et al., 1997]. After receiving alkylating regimens, MSI is developed in peripheral blood mononuclear cells in 90% breast cancer patients [Fonseca et al., 2005]. Ovarian cancer patients with MSI-negative primary resected tumors acquire MSI in their residual tumors post cisplatin-based chemotherapy [Watanabe et al., 2001c]. After successful chemotherapy, MSI is frequently found in secondary cancers such as therapy-related acute myeloid leukemia/myelodysplastic syndrome (t-AML/MDS). The incidence of MSI in t-AML/MDS ranges from 20% to 94% of such cases, significantly higher than that in de-novo AML (<5% MSI) [Rund et al., 2005; Das-Gupta et al., 2001; Sheikhha et al., 2002; Casorelli et al., 2003].

Chemotherapeutic agents are classified by their distinct mechanisms of action. Anti-metabolites such as 5-fluorouracil (5-FU) and methotrexate interfere with DNA biosynthesis. Alkylating agents such as N-(2-Chloroethyl)-N′-cyclohexyl-N-nitrosourea (CCNU) and oxaliplatin cause base modifications and DNA strand crosslinks. It is noteworthy that oxaliplatin is a platinum-based alkylating-like agent since it does not have an alkyl group, but damages DNA in a similar way as alkylating agents. Topoisomerase inhibitors such as etoposide interrupt with DNA replication and transcription by altering DNA supercoiling. On the other hand, spindle poisons such as vinblastine do not target DNA and instead affect microtubule dynamics, hence mitosis. Since chemotherapy is generally given in drug combinations, the MSI-inducing ability of individual drugs and strategies for preventing drug-induced MSI remain poorly understood.

### SUMMARY OF THE INVENTION

Broadly, the present invention is based on a novel therapeutic approach for preventing the development of MSI-associated drug resistance and secondary cancer in cancer patients during and post chemotherapy by utilizing antioxidants that suppress drug-induced MSI but do not decrease the drug's cytotoxicity. These results are based on exemplary experiments involving colorectal cancer which frequently has germline or somatic defects in DNA mismatch repair (MMR), a system that normally repairs replicative errors and DNA adducts upon exposure to DNA-damaging agents. The work is based on experiments using a human colorectal cancer HCT116 cell line, which is MMR-deficient owing to a homozygous mutation of the hMLH1 gene, and an isogenic HCT116+chr3 cell line, which is MMR-proficient because of the transfer of chromosome 3 containing a wild-type hMLH1 gene to HCT116. In the work leading to the present invention, a sensitive and reliable in-vivo MSI reporter was employed to rapidly monitor the MSI status of drug-treated cells with a view to the design of a new strategy for preventing the development of MSI-associated drug resistance and secondary cancer in cancer patients. This work demonstrated that 5-fluorouracil, CCNU, methotrexate, etoposide, vinblastine and oxaliplatin individually induced MSI in HCT116 cells. This MSI induction occurred concomitantly with decreased steady-state levels of MMR proteins and increased intracellular levels of reactive oxygen species (ROS), suggesting that MMR deficiency and ROS are contributing factors to drug-induced MSI. An initially functional MMR system in HCT116+chr3 cells, however, readily suppressed 65-96% of drug-induced MSI seen in MMR-deficient HCT116 cells. This indicates a crucial role of MMR in minimizing drug-induced MSI. Previously, this inventor and colleagues reported that ROS such as H₂O₂ not only inactivate the MMR function but also increase the MSI frequency and thiol compounds such as N-acetylcysteine (NAC) and glutathione are potent suppressors of MSI induced by oxidative stress. Moreover, this work demonstrated that certain tested antioxidants enhanced drug's cytotoxicity while others decreasing it, indicating it is necessary to screen a larger numbers of antioxidants to target drug-generated ROS, hence MSI. By focusing on oxaliplatin-induced MSI and oxaliplatin-mediated cytotoxicity, the work has provided a method for identifying MSI-modulating compounds suitable for the use in high-throughput screening of compound libraries. The work disclosed herein also shows that antioxidants, such as gossypol, are able to suppress oxaliplatin-induced MSI while enhancing oxaliplatin-mediated cytotoxicity at the therapeutically effective amounts. Given the link between MSI and chemotherapy, these antioxidants suggest a novel therapeutic approach for preventing MSI-associated drug resistance and secondary cancer in cancer patients.

Accordingly, in a first aspect, the present invention provides a method for reducing microsatellite instability in chemotherapy, which comprises administering a therapeutically effective amount of an antioxidant to an individual receiving the chemotherapy.

In a further aspect, the present invention provides a method for reducing microsatellite instability in chemotherapy, comprising administering a therapeutically effective amount of an antioxidant to an individual receiving the chemotherapy, in which the antioxidant enhances the cytotoxicity of a chemotherapeutic agent(s) given to the individual.

In a further aspect, the present invention provides a method for reducing microsatellite instability in chemotherapy, while the chemotherapy is performed by administering a chemotherapeutic agent includes, but not limited to, drug classes of anti-metabolites, alkylating agents, topoisomerase II poisons, microtubule disruptors, their derivatives, or a combination thereof.

Specifically, said chemotherapeutic agent is selected from the group consisting of 5-fluorouracil, lomustine (CCNU), methotrexate, etoposide, vinblastine, oxaliplatin, their derivatives, and a combination thereof.

In a further aspect, the present invention provides a method for reducing microsatellite instability in chemotherapy by administering a therapeutically effective amount of an antioxidant to an individual receiving the chemotherapy, and the antioxidant includes, but not limited to, classes of phenolic antioxidants, flavone antioxidants, and/or hydroxyl radical scavengers, and/or derivatives thereof.

Specifically, said antioxidant is selected from the group consisting of CDC, ciclopirox ethanolamine, gossypol, n-octyl caffeate, baicalein, curcumin, their derivatives, and a combination thereof.

In a further aspect, the present invention provides a pharmaceutical composition comprising at least one chemotherapeutic agent and at least one antioxidant. The chemotherapeutic agent includes, but not limited to, drug classes of anti-metabolites, alkylating agents, topoisomerase II poisons, microtubule disruptors, their derivatives, and a combination thereof. The antioxidant includes, but not limited to, classes of phenolic antioxidants, flavone antioxidants, and/or hydroxyl radical scavengers, and/or derivatives thereof. In a preferred embodiment, the chemotherapeutic agent(s) is selected from the group consisting of 5-fluorouracil, lomustine (CCNU), methotrexate, etoposide, vinblastine, oxaliplatin, their derivatives, and a combination thereof, while the antioxidant is selected from the group consisting of CDC, ciclopirox ethanolamine, gossypol, n-octyl caffeate, baicalein, curcumin, their derivatives, and a combination thereof.

The full names, structures and database accession information for preferred antioxidants and chemotherapeutic agents are as follows:

CDC (a phenolic antioxidant)

IUPAC Name: [(E)-3-phenylprop-2-enyl] (Z)-2-cyano-3-(3,4-dihydroxyphenyl)prop-2-enoate

CAS: 132465-11-3

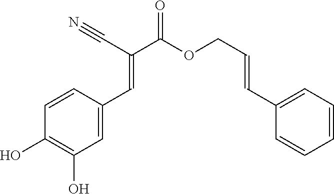 [SMILES: N#C/C(=C\c1ccc(O)c(O)c1)C(=O)OC/C=C/c1ccccc1]

Ciclopirox Ethanolamine (a Hydroxyl Radical Scavenger)

IUPAC Name: 2-aminoethanol;6-cyclohexyl-1-hydroxy-4-methylpyridin-2-one

CAS: 41621-49-2

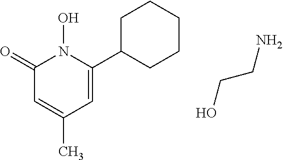

Gossypol (a Phenolic Antioxidant)

IUPAC Name: 7-(8-formyl-1,6,7-trihydroxy-3-methyl-5-propan-2-ylnaphthalen-2-yl)-2,3,8-trihydroxy-6-methyl-4-propan-2-ylnaphthalene-1-carbaldehyde

CAS: 303-45-7

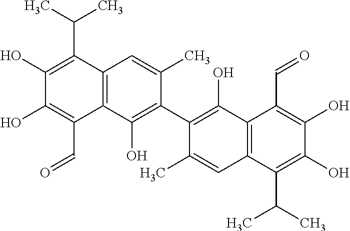 [SMILES: Cc1cc2c(C(C)C)c(O)c(O)c(C=O)c2c(O)c1-c1c(C)cc2c(C(C)C)c(O)c(O)c(C=O)c2c1O]

n-Octyl Caffeate (a Phenolic Antioxidant)

IUPAC Name: Octyl 3-(3,4-dihydroxyphenyl)prop-2-enoate

CAS: NA

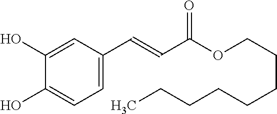 [SMILES: CCCCCCCCOC(=O)/C=C/c1ccc(O)c(O)c1]

Baicalein (a Flavone Antioxidant)

IUPAC Name: 5,6,7-trihydroxy-2-phenylchromen-4-one

CAS: 491-67-8

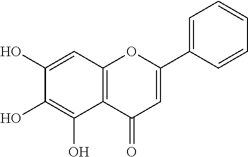 [SMILES: O=c1cc(-c2ccccc2)oc2cc(O)c(O)c(O)c12]

Curcumin (a Phenolic Antioxidant)

IUPAC Name: (1E,6E)-1,7-bis(4-hydroxy-3-methoxyphenyl)hepta-1,6-diene-3,5-dione

CAS: 458-37-7

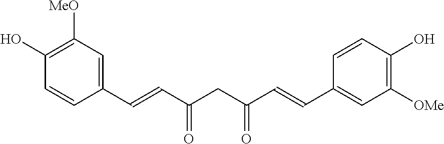 [SMILES: COc1cc(/C=C/C(=O)CC(=O)/C=C/c2ccc(O)c(OC)c2)ccc1O]

Oxaliplatin (an Alkylating Agent)

IUPAC Name: (1R,2R)-cyclohexane-1,2-diamine;oxalic acid;platinum

CAS: 53121-00-6 and 61825-94-3

5-Fluorouracil

IUPAC Name: 5-fluoro-1H-pyrimidine-2,4-dione

CAS: 51-21-8

CCNU

IUPAC Name: 1-(2-chloroethyl)-3-cyclohexyl-1-nitrosourea

CAS: 13010-47-4

Methotrexate

IUPAC Name: (2S)-2-[[4-[(2,4-diaminopteridin-6-yl)methyl-methylamino]benzoyl]amino] pentanedioic acid

CAS: 59-05-2

Etoposide

IUPAC Name: (5S,5aR,8aR,9R)-5-[[(2R,4aR,6R,7R,8R,8aS)-7,8-dihydroxy-2-methyl-4,4a,6,7,8,8a-hexahydropyrano[3,2-d][1,3]dioxin-6-yl]oxy]-9-(4-hydroxy-3,5-dimethoxyphenyl)-5a,6,8a,9-tetrahydro-5H-[2]benzofuro[6,5-f][1,3]benzodioxol-8-one

CAS: 33419-42-0

Vinblastine

IUPAC Name: dimethyl (2β3β,4β,5α,12β,19α)-15-[(5S,9S)-5-ethyl-5-hydroxy-9-(methoxycarbonyl)-1,4,5,6,7,8,9,10-octahydro-2H-3,7-methanoazacycloundecino[5,4-b]indol-9-yl]-3-hydroxy-16-methoxy-1-methyl-6,7-didehydroaspidospermidine-3,4-dicarboxylate

CAS: 865-21-4

In a further aspect, the present invention provides a method of screening for compounds useful in reducing drug-induced MSI and/or drug's cytotoxicity, the method employing first and second cell lines, wherein the first cell line is deficient in a component of the DNA mismatch repair (MMR) system and the second cell line is proficient for DNA mismatch repair (MMR) system that harbor a dual-fluorescent MSI reporter, the method comprising:

(a) contacting the first line with at least one candidate antioxidant with a chemotherapeutic agent;

(b) determining the MSI frequency and/or amount of cell death in the first cell line;

(c) selecting a promising candidate antioxidant which suppresses drug-induced MSI and/or enhancing drug's cytotoxicity in the first cell line;

(d) determining the MSI frequency and/or amount of cell death in the second cell line when contacting the promising candidate antioxidant with a chemotherapeutic agent; and
(e) selecting a promising candidate antioxidant which suppresses drug-induced MSI or enhancing drug's cytotoxicity in the second cell line.

In this method, it is preferable that the first and second cells lines are isogenically matched. It is also preferred that the cell lines are cancer cell lines, for example a human colorectal cancer cell line, such as HCT116 used in the examples. The use of human cell lines or those from animal models (e.g. murine or zebrafish) are preferred.

As set out in detail below, candidate antioxidants identified using a method of screening according to the present invention may be the subject of further development to optimize their properties, to determine whether they work well in combination with other chemotherapy or radiotherapy, to manufacture the agent in bulks and/or to formulate the agent as a pharmaceutical composition.

Embodiments of the present invention will now be described in more detail by way of example and not limitation with reference to the accompanying figures.

### BRIEF DESCRIPTION OF THE DRAWINGS

FIG. 1A to FIG. 1M. Effects of chemotherapeutic agents on the viability and microsatellite instability (MSI) of MMR-deficient HCT116 derivatives. HCT116-(CA)₁₃ and HCT116-(N)₁₆ cells, harboring the (CA)₁₃ reporter microsatellite and (N)₁₆ random sequence, respectively, in the dual fluorescent reporter were treated with a specified drug at various concentrations for three days. (FIG. 1A-FIG. 1F) At the end of drug treatment, cell viability was determined by MTT assay and expressed as the optical density of drug-treated cells, relative to that of untreated cells. (FIG. 1G-FIG. 1L) After a 3-day recovery from drug treatment, the frameshift mutation frequency was analyzed by flow cytometry and expressed as the percentage of the GFP⁺RFP⁺ dual-fluorescent subpopulation in the GFP⁺ single-fluorescent cell population. Data are expressed as means±SD. Relative to untreated control, statistical significance is indicated by * (P<0.05), ** (P<0.005) or *** (P<0.0005). All tested drugs increased the mutations in the (CA)₁₃ microsatellite and, to a lesser degree, the (N)₁₆ random sequence in a dose-dependent manner. (FIG. 1M) HCT116-(CA)₁₃ cells were treated with a chemotherapeutic agent at a specified concentration for one day (1d) or three days (3d), followed by a 3-day recovery. Untreated cells served as the control. Genomic DNA of untreated and treated cells was analyzed for the instability of endogenous microsatellites by fluoresceinated PCR-based assay with the Bethesda panel of microsatellite markers, commonly used for diagnosing the MSI status of cancer patients. Based on the fragment size in base pairs (bp) in electropherograms, an insertion(s) or deletion(s) of the repeat unit in the microsatellite sequence is indicated by a right- or left-pointed arrow, respectively. A grey arrowhead indicates a 1-bp insertion in a non-repetitive region of the D5S346 microsatellite.

FIG. 2A to FIG. 2C. Effects of chemotherapeutic agents on the viability, MSI and MMR protein levels of human colorectal cancer cells. MMR-deficient HCT116 and MMR-proficient HCT116+chr3 cells, as well as their derivatives harboring a (CA)₁₃ reporter microsatellite, were treated for three days with 10 μM 5-FU, 50 μM CCNU, 25 nM methotrexate (MTX), 5 μM etoposide (ETO), 25 nM vinblastine (VBL) or 1 μM oxaliplatin (L-OHP). Untreated cells served as the control (Ctl). (FIG. 2A) At the end of the drug treatment, cell viability was analyzed by MTT assay. No significant differences in response to drug's cytotoxicity were detectable between MMR-deficient and MMR-proficient cells under the treatment conditions. After a 3-day recovery from the drug treatment, (FIG. 2B) MSI was analyzed by high-content microscopy and expressed as the frameshift mutation frequency, which is defined as the percentage of the DsRed⁺GFP⁺Hoechst⁺ subpopulation in the GFP⁺Hoechst⁺ population. In addition, (FIG. 2C) the levels of specified MMR proteins were analyzed by Western blotting, and actin served as the loading control.

FIG. 3. Effects of N-acetylcysteine (NAC) on drug-generated ROS in HCT116 cells. HCT116 cells were treated with 10 μM 5-FU, 50 μM CCNU, 25 nM methotrexate (MTX), 5 μM etoposide (ETO), 25 nM vinblastine (VBL) or 1 μM oxaliplatin (L-OHP), in the presence or absence of 2.5 mM NAC for one day (1d-T), three days (3d-T) or three days plus a 3-day recovery (3d-T+3d-R). Intracellular reactive oxygen species (ROS) levels were determined with a 2′,7′-dichlorodihydrofluorescein diacetate (DCDHF-DA) based assay by flow cytometry and expressed as the mean fluorescence intensity (MFI) of 2′,7′-dichlorofluorescein (DCF). Untreated cells served as the control (Ctl). A statistical difference between drug-treated cells with or without NAC at the same time point is indicated by * (P<0.05) or *** (P<0.0005).

FIG. 4A to FIG. 4L. Effects of five antioxidants on drug-generated ROS, drug-induced MSI and viability of HCT116 and derivatives. HCT116 cells were allowed to recover for three days from a 3-day treatment of 10 μM 5-FU, 50 μM CCNU, 25 nM methotrexate (MTX), 5 μM etoposide (ETO), 25 nM vinblastine (VBL) or 1 μM oxaliplatin (L-OHP). Untreated cells served as the control (Ctl). Intracellular ROS levels in the cells were determined with a DCDHF-DA based assay by flow cytometry, and expressed as the mean fluorescence intensity (MFI) of DCF. HCT116-(CA)₁₃ cells were co-treated for three days with a specified drug and one of five antioxidants, including 2.5 mM NAC, 5 mM glutathione (GSH), 250 μM vitamin (Vit) C, 1.5 μM curcumin (Cur) and 150 μM eugenol (Eug). The control (Ctl) denotes untreated cells. (FIG. 4A, FIG. 4C, FIG. 4E, FIG. 4G; FIG. 4I, FIG. 4K) After 3-day recovery from the co-treatment, MSI was analyzed by high-content microscopy and expressed as frameshift mutations in HCT116-(CA)₁₃ cells. (FIG. 4B, FIG. 4D, FIG. 4F, FIG. 4H, FIG. 4J, FIG. 4L) At the end of co-treatment, the cell viability was analyzed by the MTT assay and expressed as the optical density (OD) at 595 nm. Data are expressed as means±SD from representative experiments. A statistical difference between drug-treated cells with a specified antioxidant and without an antioxidant (none) is indicated by * (P<0.05), ** (P<0.005) and *** (P<0.0005).

FIG. 5A to FIG. 5E. Screen of a REDOX library for compounds that suppress oxaliplatin-induced MSI without compromising the cytotoxicity of oxaliplatin. (FIG. 5A) Eighty four compounds in the REDOX library are grouped by their known functions. HCT116-(CA)₁₃ cells were co-treated with 1 μM oxaliplatin (L-OHP) and a low or a 10-fold higher concentration of a compound. After a 3-day co-treatment followed by a 3-day recovery, both the MSI frequency and cell numbers (e.g., cytotoxicity) in the cells were simultaneously analyzed by high-content microscopy. Relative to the treatment with oxaliplatin alone, (FIG. 5B) ˜90% of compounds in the library suppressed oxaliplatin-induced MSI (in the light blue wedge), and (FIG. 5C) ˜34% of compounds enhanced oxaliplatin-mediated cytotoxicity (in the light green wedge). The quality of the assay is assessed by Z′-factor, where 1>Z′-factor≧0.5 indicates an excellent assay. (FIG. 5D and FIG. 5E) When the MSI frequency and cell numbers were plotted together, compounds that are located in the lower left quarter and perhaps in the lower right quarter are potential candidates. Red dotted lines, set at 100%, indicate the MSI frequency and numbers of the cells treated with oxaliplatin alone.

FIG. 6A to FIG. 6F. Effects of antioxidant candidates on oxaliplatin-induced MSI and cytotoxicity in HCT116 derivatives. HCT116-(CA)₁₃ cells were co-treated with 1 μM oxaliplatin and specified concentrations of (FIG. 6A) CDC, (FIG. 6B) ciclopirox ethanolamine, (FIG. 6C) gossypol, (FIG. 6D) n-octyl caffeat, (FIG. 6E) baicalein or (FIG. 6F) curcumin for three days followed by a 3-day recovery. Relative cell numbers (in black bars) and MSI frequency (in grey bars) were simultaneously analyzed by high-content microscopy. The MSI frequency or cell numbers of cells treated with oxaliplatin alone is set at 100%, as indicated by the dotted green line.

FIG. 7. Gossypol and curcumin display similar effects on the cytotoxicity of oxaliplatin cytotoxicity between MMR-deficient and MMR-proficient cells. MMR-deficient HCT116 and MMR-proficient HCT116+chr3 cells were co-treated for three days with 1 μM oxaliplatin and specified concentrations of (A, B) gossypol and (C, D) curcumin. Untreated cells served as the control (Ctl). With (black lines) or without (blue lines) a 3-day recovery from a 3-day co-treatment, cell viability was analyzed by MTT assay. Gossypol, but not curcumin, enhanced the cytotoxicity of oxaliplatin in a dose-dependent manner.

### DETAILED DESCRIPTION OF THE PREFERRED EMBODIMENT

1. Materials and Methods

1.1 Chemicals and reagents

5-Fluorouracil, CCNU, methotrexate, etoposide, vinblastine, oxaliplatin, dimethyl-sulfoxide (DMSO), the β-actin specific antibody, species-specific IgG conjugated with horseradish peroxidase, and 2′,7′-dichlorofluorescin diacetate (DCF-DA) were purchased from Sigma-Aldrich (St. Louis, Mo., USA). Dulbecco's Modified Eagle's Medium with F-12 nutrient mixture (DMEM/F-12) and fetal bovine serum (FBS) were obtained from Hyclone (Logan, Utah, USA). L-glutamine, 0.25% trypsin, G418, hygromycin, Lipofectamine 2000™, an hMSH6-specific antibody and PCR primers with or without fluorescent labeling were purchased from Invitrogen (Grand Island, N.Y., USA). The EasyPure Genomic DNA mini Kit was purchased from Bioman Scientific (Taipei, Taiwan). The polyvinylidene difluoride membrane and the chemiluminescent detection kit were from Millipore (Billerica, Mass., USA), and antibodies specific for hMLH1 and hMSH2 came from BD (Franklin Lakes, N.J., USA).

1.2 Cell Culture of HCT116 and Derivatives

The HCT116 cell line from American Type Culture Collection (ATCC, Manassas, Va., USA) was maintained at 5% CO₂ and 37° C. in the growth medium (DMEM/F-12 containing 10% FBS and 2 mM L-glutamine). Derived from HCT116, the HCT116-(CA)₁₃ and HCT116-(N)₁₆ stable transfectants harbor a dual-fluorescent reporter containing the (CA)₁₃ microsatellite and a random (N)₁₆ sequence respectively [Li et al., 2014]. These transfectants were cultured in the growth medium supplemented with 200 μg/ml hygromycin. HCT116-derived and MMR-proficient HCT116+chr3 cells [Koi et al., 1994], kindly provided by C. R. Boland, harboring a (CA)₁₃ microsatellite in the dual-fluorescent reporter were cultured in the growth medium containing 200 μg/ml hygromycin and 400 μg/ml G418. All cells were evaluated by their morphology and by Western blot analysis of MMR gene products, as well as by STR DNA profiling (performed by the NCKU DNA Sequencing Core).

1.3 Drug Treatment with or without an Antioxidant

Specified cells were seeded at 1×10⁴ or 1×10⁵ cells per well in 96-well or 12-well plates respectively. One day after seeding, the cells were treated in triplicates for 3 days with a tested drug in the presence or absence of an antioxidant at indicated concentrations. Subsequently, the drug and/or antioxidant were removed and cells were washed and recovered in fresh growth medium for 3 days before being subjected to analyses. Solvents used for making the stock solution of drugs include ethanol for 5 mM CCNU stock, 0.1 mM NaOH for 1 mM methotrexate, saline (0.9% NaCl) for 50 mM 5-FU, 5% dextrose for 5 mM oxaliplatin, and DMSO for 42.5 mM etoposide and 5.5 mM vinblastine stocks. On the other hand, PBS was used to make 0.5 M vitamin C stock, saline for making 0.5 M NAC and 0.1 M GSH stocks, and DMSO for making 10 mM curcumin and 10 mM eugenol. Prior to the experiments, PBS was used for diluting the drugs and antioxidants and the final solvent concentration was kept the same for all tested compounds, including untreated controls.

1.4 REDOX Library Screen by High-Content Microscopy

The REDOX library (Cat# BML-2835, ENZO, USA) is composed of 84 compounds that were supplied at 10 mM in DMSO. HCT116-(CA)₁₃ cells were seeded at 1×10⁴ cells per well in 96-well glass plates containing 100 μl growth medium. One day after seeding, the cells were co-treated with 1 μM oxaliplatin and an antioxidant at specified concentrations in triplicate for three days, followed by a 3-day recovery in fresh growth medium without the drug and antioxidants. Subsequently, the cells were fixed with 4% paraformaldehyde and nuclei were stained with 1 μg/ml Hoechst 33258 before being analyzed for MSI and cell number by high-content fluorescence microscopy.

1.5 Cell Viability Assay

Cell viability was determined by the MTT assay. Briefly, the optical density of colored formazan converted from MTT by viable cells was determined as previously described [Chang et al., 2002] at 595 nm with an ELISA reader (Thermo Labsystems). Cell viability is expressed as the optical density value of drug-treated cells relative to that of solvent-treated cells, after subtracting the background from the medium.

1.6 Frameshift Mutation Analysis by Flow Cytometry

At the end of treatment, adherent cells in 12-well plates were trypsinized and resuspended in PBS containing 1 mM EDTA, and filtered through a 40-μm strainer, and then subjected to flow cytometry (Quanta™ SC-MPL, Beckman Coulter). As detailed previously [Li et al., 2014], a minimum of 1×10⁴ GFP⁺ cells were analyzed per sample and displayed on green fluorescence (FL1) versus red fluorescence (FL2) axes using Quanta SC MPL Analysis software. The frequency of frameshift mutations is expressed as the percentage of DsRed⁺GFP⁺ subpopulation in the GFP⁺ cell population.

1.7 Microsatellite Mutation Analysis by High-Content Fluorescent Microscopy

After treatment, HCT116-(CA)₁₃ cells in 96-well glass plates (Corning-Costar®) were fixed with 4% paraformaldehyde and nuclei were stained with 1 μg/ml Hoechst 33258. Fluorescent images of the cells were acquired at 100× magnification by ImageXpress^{Micro} system (Molecular Devices) and analyzed with MetaXpress® V3.1 software as described previously [Li et al., 2014]. The frequency of frameshift mutations of the (CA)₁₃ microsatellite is expressed as the percentage of the DsRed⁺GFP⁺Hoechst⁺ subpopulation in the GFP⁺Hoechst⁺ population.

1.8 Microsatellite Mutation Analysis by Fluorescinated PCR-Based Assay

Genomic DNA was isolated from HCT116 derivatives in 12-well plates with EasyPure Genomic DNA mini kit. The isolated DNA were amplified with fluoresceinated primers specific for the Bethesda panel of microsatellite [Boland et al., 1998] and selected coding microsatellites as described previously [Li et al., 2014]. Fluoresceinated PCR products were analyzed by the ABI 310 genetic analyzer, and electropherograms were generated with GeneScan Collection software (ABI).

TABLE 1
Primers for fluorescinated PCR-based MSI assay

| | | | Size of PCR | |
|---|---|---|---|---|
| Primer | GDB # | Sequence | products | Microsatellite |
| BAT25-F | 9834508 | 5′-(*Hex)TCGCCTCCAAGAATGTAAGT | 124 bp | TTTT.T.TTTT.(T)₇.A(T)₂₅ |
| BAT25-R | | 5′- TCTGCATTTTAACTATGCCTC | | |
| BAT26-F | 9834505 | 5′-(*Tet)TGACTACTTTTGACTTCAGCC | 122 bp | (T)₅ . . . (A)₂₆ |
| BAT26-R | | 5′-AACCATTCAACATTTTTAACCC | | |
| D17S250-F | 177030 | 5′-(*Fam)GGAAGAATCAAATAGACAAT | 150 bp | (TA)₇ . . . (CA)₂₄ |
| D17S250-R | | 5′-GCTGGCCATATATATATTTAAACC | | |
| D2S123-F | 187953 | 5′-(*Tet)AAACAGGATGCCTGCCTTTA | 220 bp | (CA)₁₃TA(CA)₁₅(T/GA)₇ |
| D2S123-R | | 5′-GGACTTTCCACCTATGGGAC | | |
| D5S346-F | 181171 | 5′-(*Fam)ACTCACTCTAGTGATAAATCG | 120 bp | (CA)₂₆ |
| D5S346-R | | 5′-AGCAGATAAGACAGTATTACTAGTT | | |

Forward and reversed primers are indicated by F and R respectively.
*indicates a specified fluorescent dye that end-labeled each forward primer, including Fluorescein-CE phosphoramidite (Fam), Hexachloro-fluorescein-CE phosphoramidite (Hex), and Tetrachloro-fluorescein-CE phosphoramidite (Tet).

1.9 the ROS Assay

Intracellular ROS levels were measured using an oxidation-sensitive 2′,7′-dichlorodihydrofluorescein diacetate (DCDHF-DA) probe, which can be oxidized to the fluorescent 2′,7′-dichloro-fluorescein (DCF) product. After treatment, HCT116 derivatives in 12-well plates were harvested, washed and resuspended in PBS containing 10 μM DCDHF-DA, followed by 30-min incubation at 37° C. in the dark before being subjected to flow cytometry (Quanta SC-MPL). For a ROS curve of H₂O₂, 2×10⁵ HCT116 derivatives were detached, washed, and incubated with 10 μM DCDHF-DA for 30 min at 37° C., followed by flow cytometric analysis after 30-min exposure to different concentrations of H₂O₂. After excluding cell debris on the basis of electronic volume and side scatter, the fluorescence intensity of DCF was measured in fluorescence channel 1 of the flow cytometer (λₑₓ: 488 nm; λₑₘ: 525 nm). The ROS level is expressed as the mean fluorescence intensity (MFI) of DCF from 10,000 cells per sample.

1.10 Western Blot Analysis

Equal amounts of total proteins in cell lysate were resolved by 8% SDS-polyacrylamide gel electrophoresis and then transferred onto a polyvinylidene difluoride membrane and MMR proteins were immunochemically and chemiluminescently detected as previously described [Chang et al., 2002].

1.11 Statistical Analysis

All experiments were performed in triplicate and repeated at least three times, and data are presented as means±SD. A difference between any treated sample and the control was assessed by 2-tailed Student's t-test. P<0.05 was considered significant.

2. Results

2.1 Chemotherapeutic Agents Preferentially Induce Mutations in the (CA)₁₃ Reporter Microsatellite.

Previously we developed a sensitive and reliable in-vivo dual-fluorescent reporter system in MMR-deficient human colorectal cancer HCT116 cells, yielding HCT116-(CA)₁₃ and HCT116-(N)₁₆ stable transfectants that harbor a (CA)₁₃ reporter microsatellite or (N)₁₆ random sequence, respectively, in the DsRed coding region [Li et al., 2014]. In these cells we examined the mutation-inducing ability of five chemotherapeutic agents (“tested drugs”, or “drug”), namely 5-FU and methotrexate (anti-metabolites), CCNU (an alkylating agent), etoposide (a topoisomerase II poison) vinblastine (a microtubule disruptor) and oxaliplatin (a platinum-based alkylating-like agent).

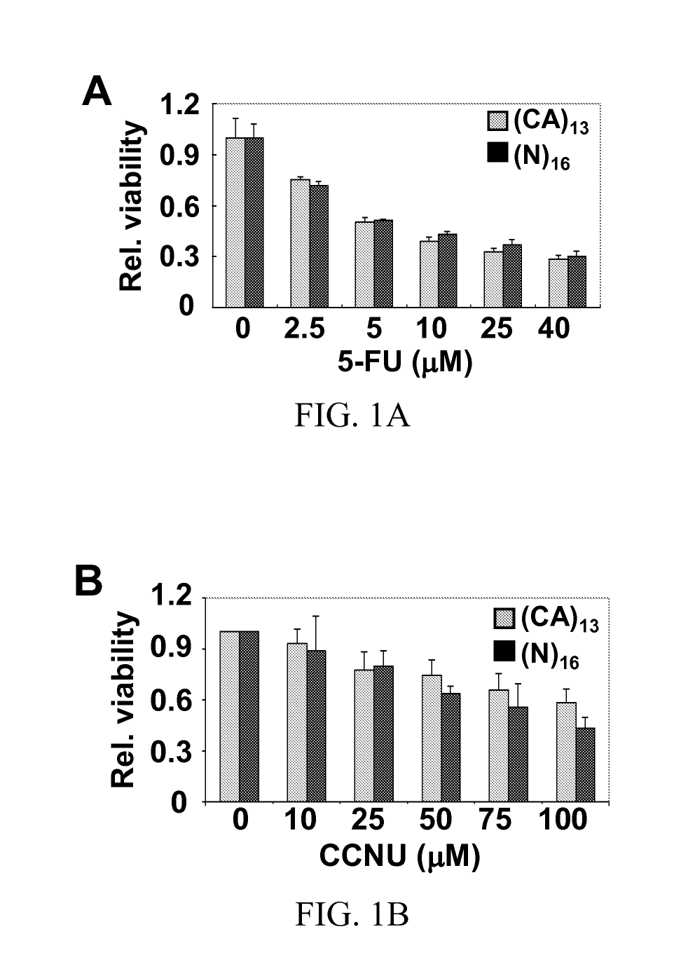

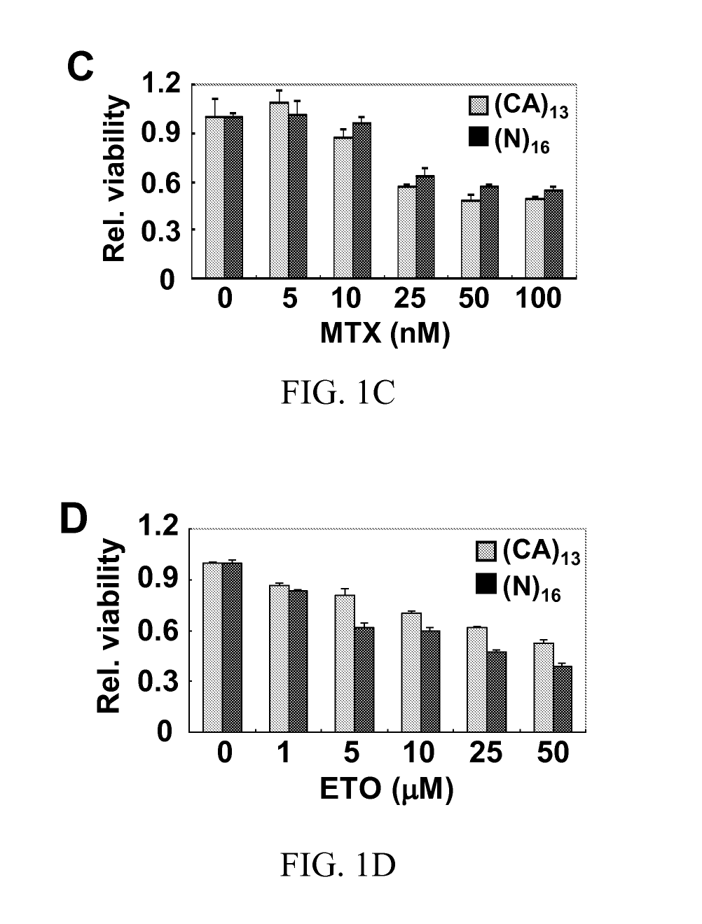

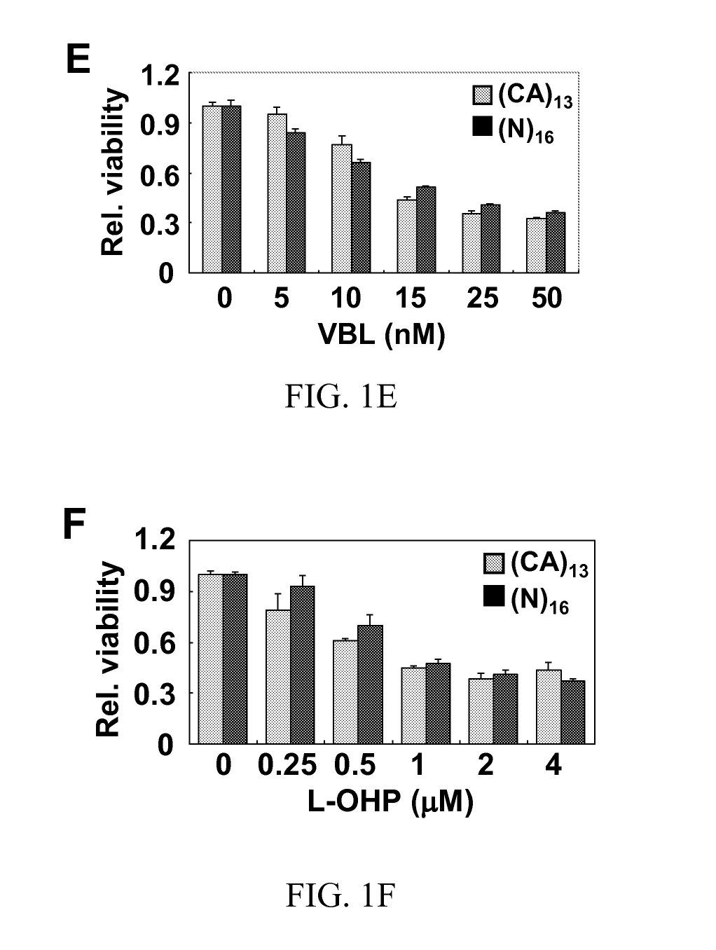

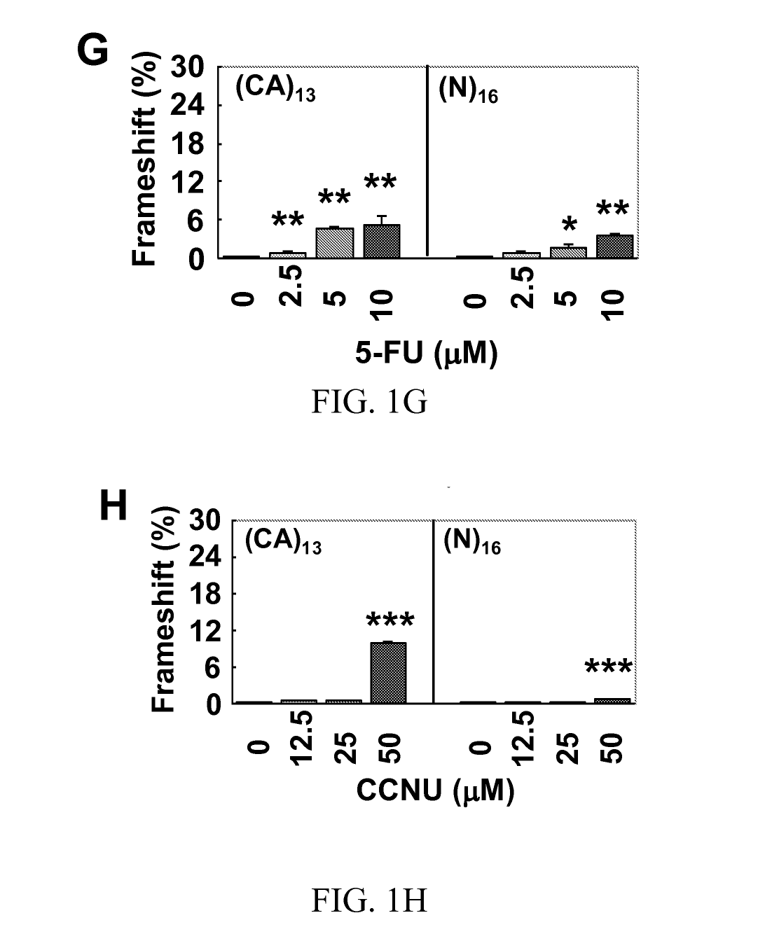

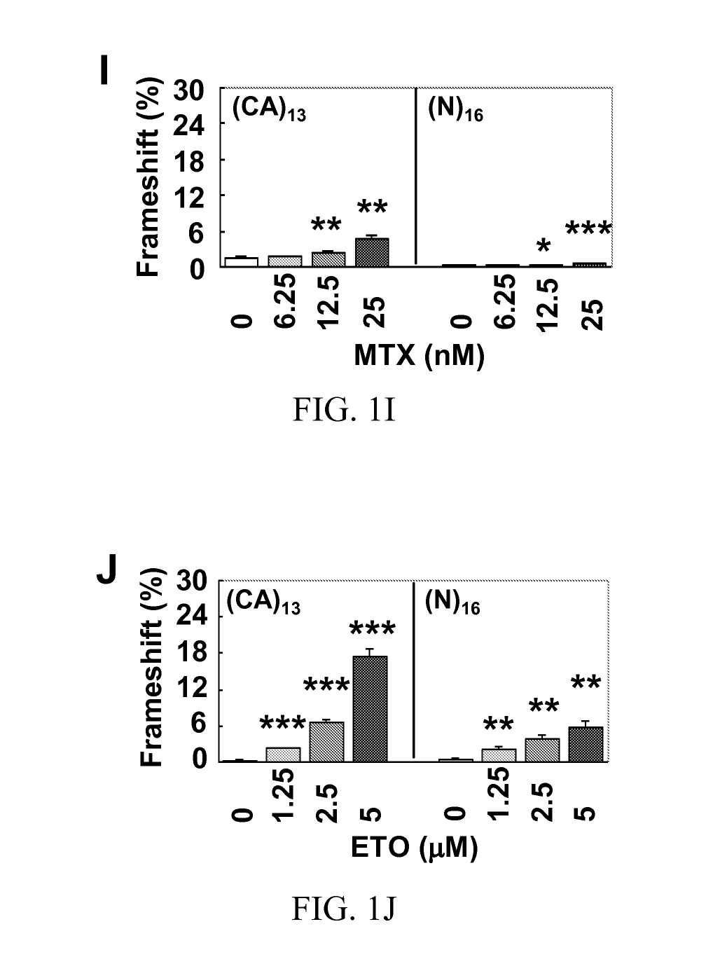

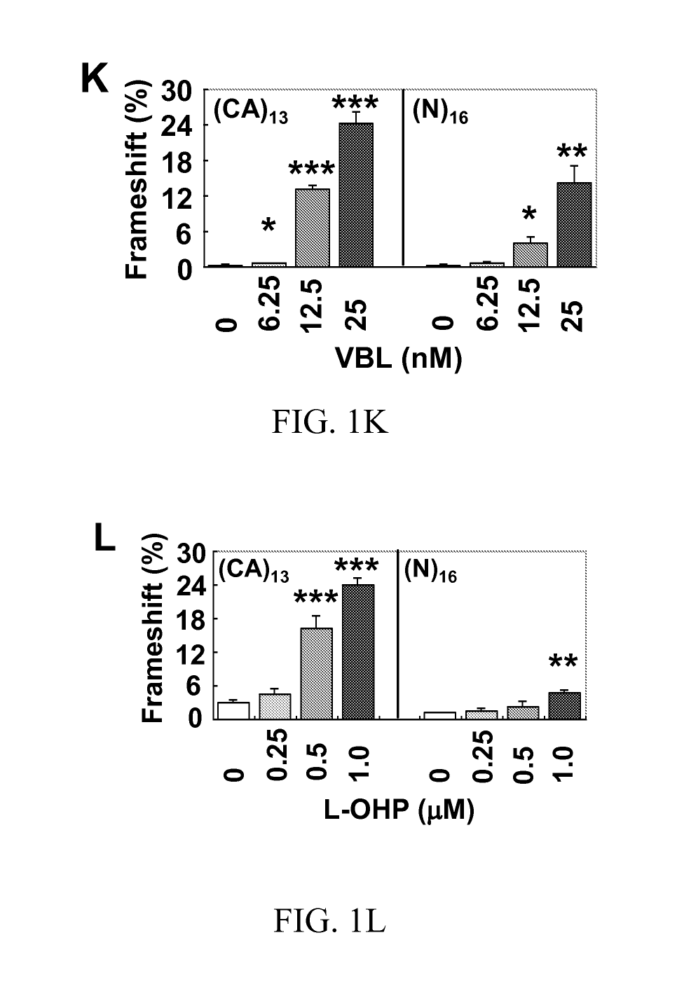

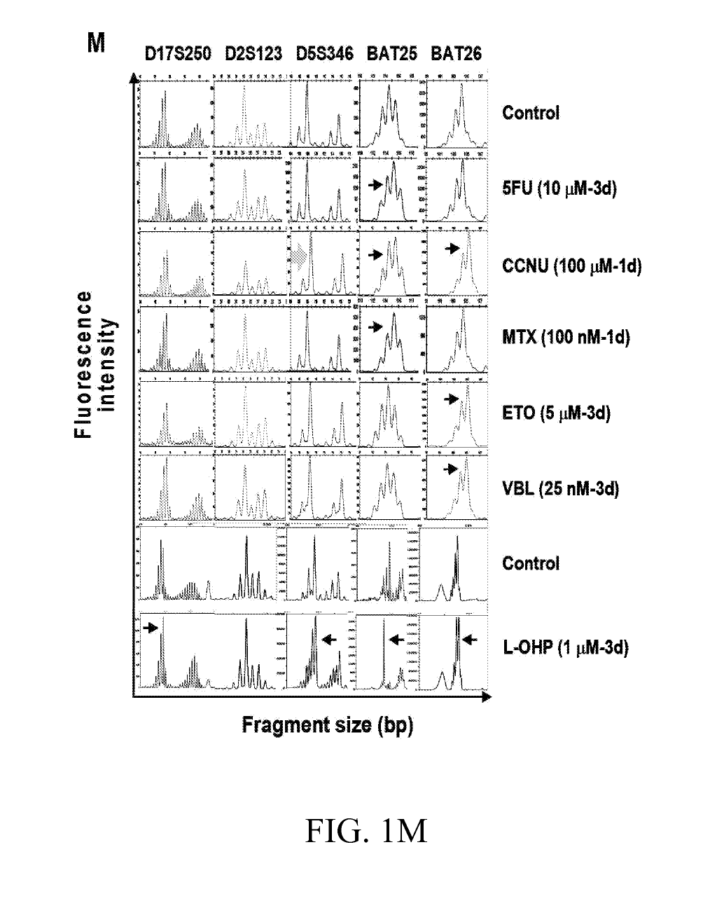

We first determined sub-lethal dose ranges of tested drugs after a 3-day treatment in HCT116 derivatives using the MTT assay (FIG. 1A-FIG. 1F). Between HCT116-(CA)₁₃ and HCT116-(N)₁₆ cells, tested drugs at specified concentrations did not show significant differences in the viability. To determine the mutation-inducing ability of tested drugs, the cells were treated with each drug at sub-lethal doses for three days followed by 3-day recovery to allow mutations to accumulate. Based on flow cytometric analysis, 2.5, 5 and 10 μM 5-FU increased the mutation frequency from 0.46±0.04% to 5.91±0.49%, 6.37±0.96% and 8.05±2.01% respectively in HCT116-(CA)₁₃ cells (FIG. 1G). Compared to the (N)₁₆ random sequence, the (CA)₁₃ microsatellite was 1.2-2.8 fold more susceptible to 5-FU-induced mutations (FIG. 1G).

The other tested drugs similarly increased the mutation (i.e., MSI) frequency in HCT116-(CA)₁₃ cells in a dose-dependent fashion as analyzed by flow cytometry (FIG. 1H-FIG. 1L). At the highest doses tested, etoposide, vinblastine and oxaliplatin induced 3-5 fold higher MSI frequency than 5-FU, CCNU and methotrexate. Relative to the (N)₁₆ random sequence, the (CA)₁₃ microsatellite was 1.7-9.1 fold more vulnerable to 50 μM CCNU, 25 nM methotrexate, 5 μM etoposide, 25 nM vinblastine and 1 μM oxaliplatin (FIG. 1G-FIG. 1L). In sum, all the tested chemotherapeutic agents from different drug classes individually increased the mutation frequency, especially in the (CA)₁₃ microsatellite sequence, in HCT116 derivatives.

2.2 Chemotherapeutic Agents Also Induce the Instability of Endogenous Microsatellites.

The Bethesda panel of microsatellite markers includes three dinucleotide repeats (D2S123, D5S346 and D17S250) and two mononucleotide repeats (BAT25 and BAT26) [Boland et al., 1998]. In a fluorescinated PCR-based assay, 10 μM 5-FU induced the instability of the BAT25 microsatellite, whereas 5 μM etoposide or 25 nM vinblastine destabilized the BAT26 microsatellite in HCT116 cells after a 3-day drug treatment and a 3-day recovery (FIG. 1M). However, we failed to detect alterations in these microsatellite markers after the cells were treated with 50 μM CCNU or 25 nM methotrexate for three days followed by a 3-day recovery (data not shown). On the other hand, a 1-day treatment with 100 μM CCNU or 100 nM methotrexate followed by a 3-day recovery caused the instability of BAT25 and/or BAT26 microsatellites (FIG. 1M). In addition, 100 μM CCNU also caused a 1-bp insertion in a non-repetitive region of D5S346 thereby shifting all the peaks in the dinucleotide repeats correspondingly (FIG. 1M). A 3-day treatment with 1 μM oxaliplatin followed by a 3-day recovery resulted in alterations of four out of five microsatellite markers in the Bethesda panel (FIG. 1M). Collectively, the tested drugs caused the instability of endogenous microsatellites in HCT116 cells that lack a functional MMR system.

2.3 A Functional MMR System Minimizes Drug-Induced MSI.

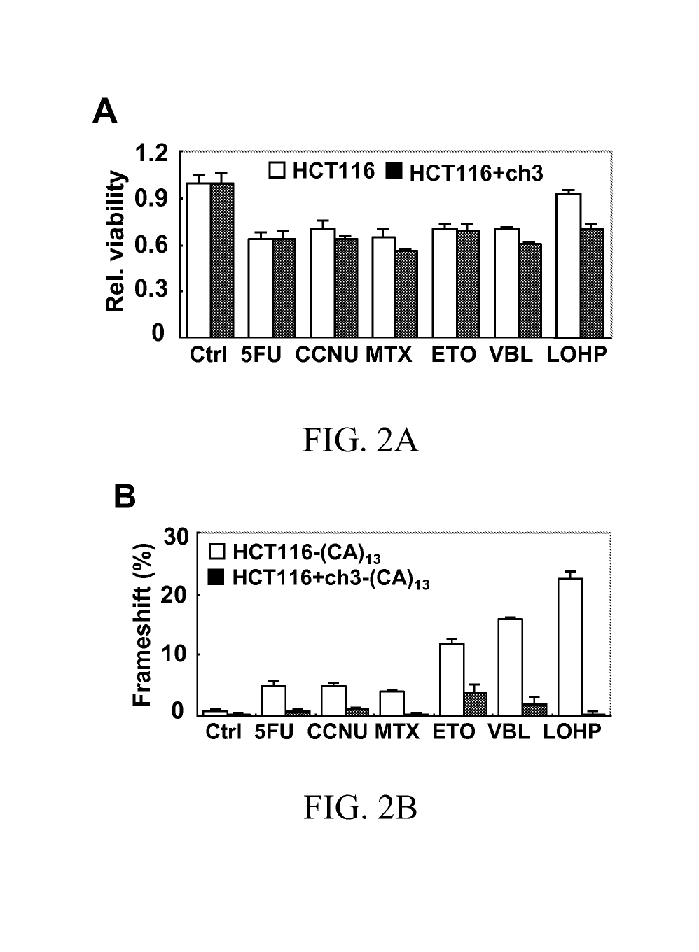

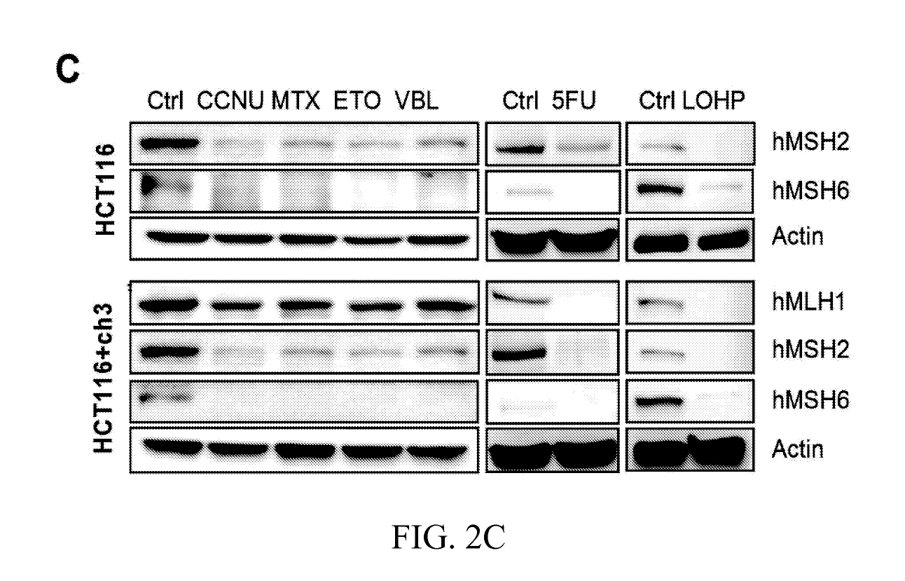

To test whether a functional MMR system protects cells from drug-induced MSI, drug effects on MMR-proficient HCT116+chr3-(CA)₁₃ [Li et al., 2014] were compared with that on MMR-deficient HCT116-(CA)₁₃ cells. Based on the MTT assay, all tested drugs had similar effects on the viability of MMR-deficient and MMR-proficient cells (FIG. 2A).

Using high-content microscopy, we found that CCNU, etoposide and vinblastine significantly increased the MSI frequency from a base line of 0.35% to 1.26% (P=0.0001), 3.74% (P=0.0039) and 2.10% (P=0.0194), respectively, in MMR-proficient HCT116+chr3-(CA)₁₃ cells (FIG. 2B). In HCT116-(CA)₁₃ cells treated with tested drugs, except anti-metabolites and oxaliplatin, the MSI frequency determined by high-content microscopy was 31-50% lower than that determined by flow cytometry. This is similar to what we previously observed in H₂O₂-treated cells, which is due to cell loss during the fixation and staining steps required for high-content microscopic analysis of MSI [Li et al., 2014]. Nevertheless, our findings indicate that a functional MMR system in HCT116+chr3 cells prevented 65-96% of drug-induced MSI seen in MMR-deficient HCT116 cells.

2.4 Chemotherapeutic Drugs Decrease Steady-State Levels of MMR Proteins

In addition to using the Bethesda panel of microsatellites, immunocytochemical analysis of MMR protein levels is also frequently used for diagnosing the MMR status of colon cancer patients [Boland et al., 1998; Umar et al., 2004]. Based on Western blot analysis, we observed that each of the tested drugs decreased steady-state levels of hMSH2 and hMSH6 proteins in MMR-proficient and MMR-deficient cells (FIG. 2C). While HCT116 cells do not express hMLH1, the tested drugs also slightly decreased the hMLH1 protein level in HCT116+chr3 cells (FIG. 2C). By decreasing steady-state levels of MMR proteins, the tested drugs likely attenuated the MMR function.

2.5 Drug-Generated ROS Contribute to Drug-Induced MSI

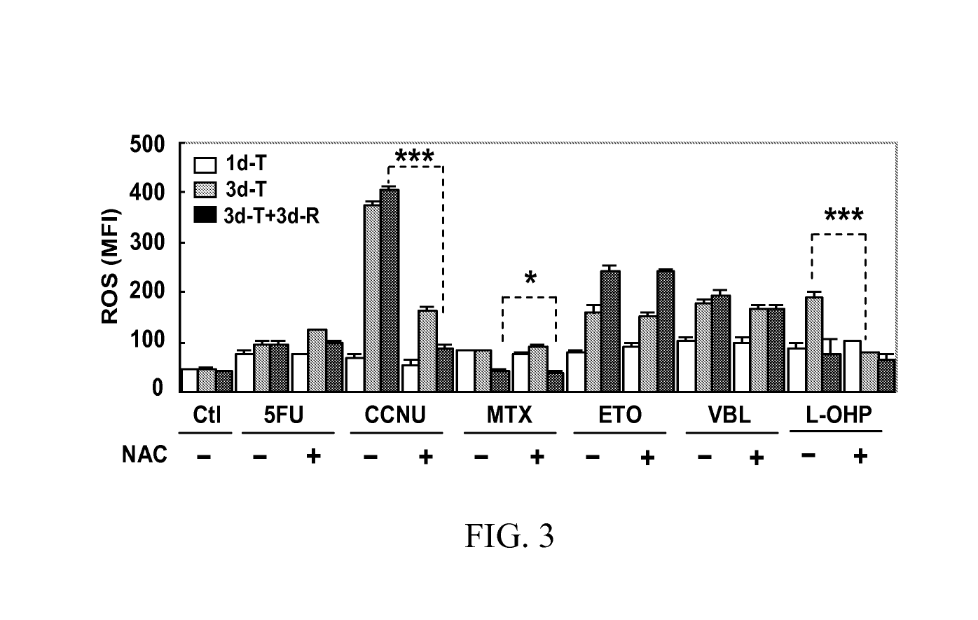

Certain chemotherapeutic agents, such as 5-FU, methotrexate and etoposide, are known to generate intracellular reactive oxygen species (ROS) [Martin et al., 2009; Hwang et al., 2001; Oh et al., 2007]. We therefore measured drug-generated ROS levels in HCT116 cells. After a 3-day recovery from a 3-day drug treatment, 10 μM 5-FU but not 25 nM methotrexate increased intracellular ROS levels (FIG. 3). Among the tested drugs, 50 μM CCNU generated the highest ROS level (FIG. 3).

Next, antioxidants were utilized to interrogate possible contributions of ROS to drug-induced MSI and cytotoxicity in HCT116-(CA)₁₃ cells by high-content microscopy and the MTT assay respectively. The doses for selected antioxidants were chosen because they exerted minimal effects on the viability and MSI of HCT116-(CA)₁₃ cells (data not shown), [Li et al., 2014].

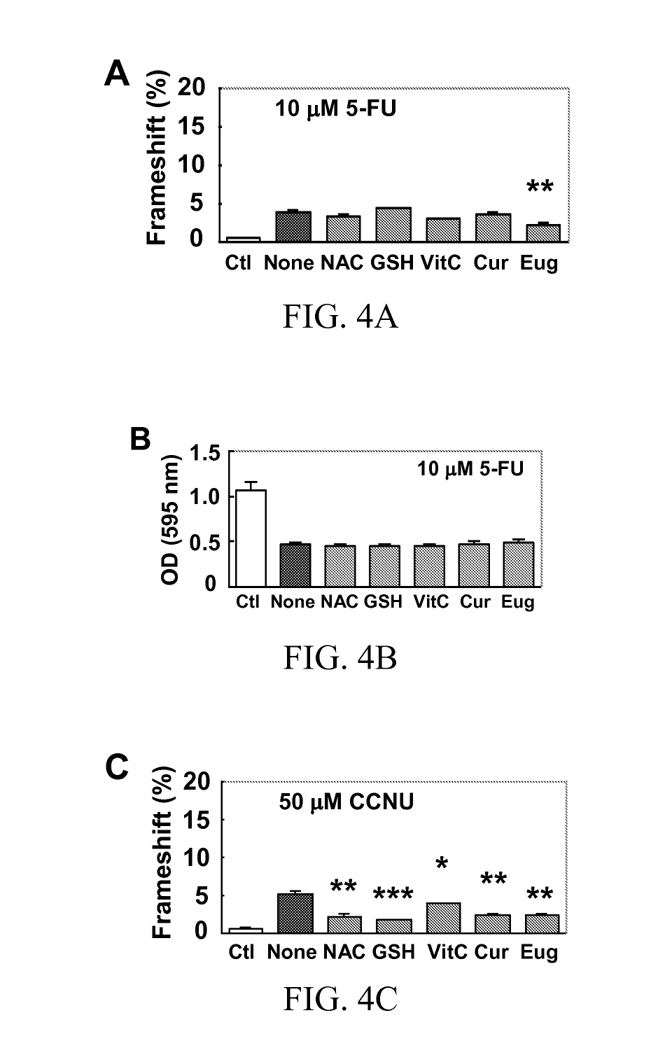

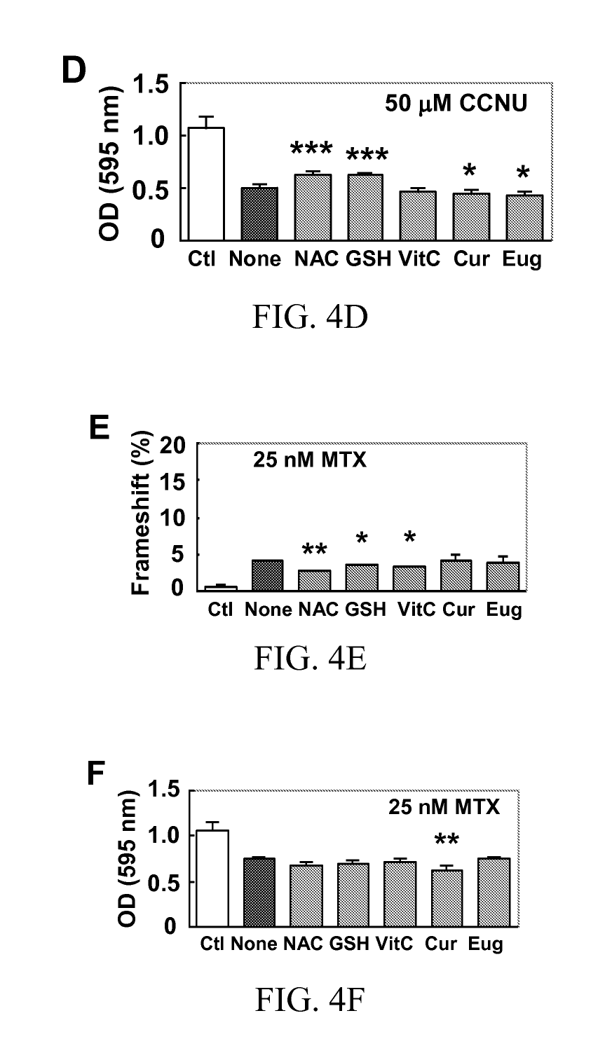

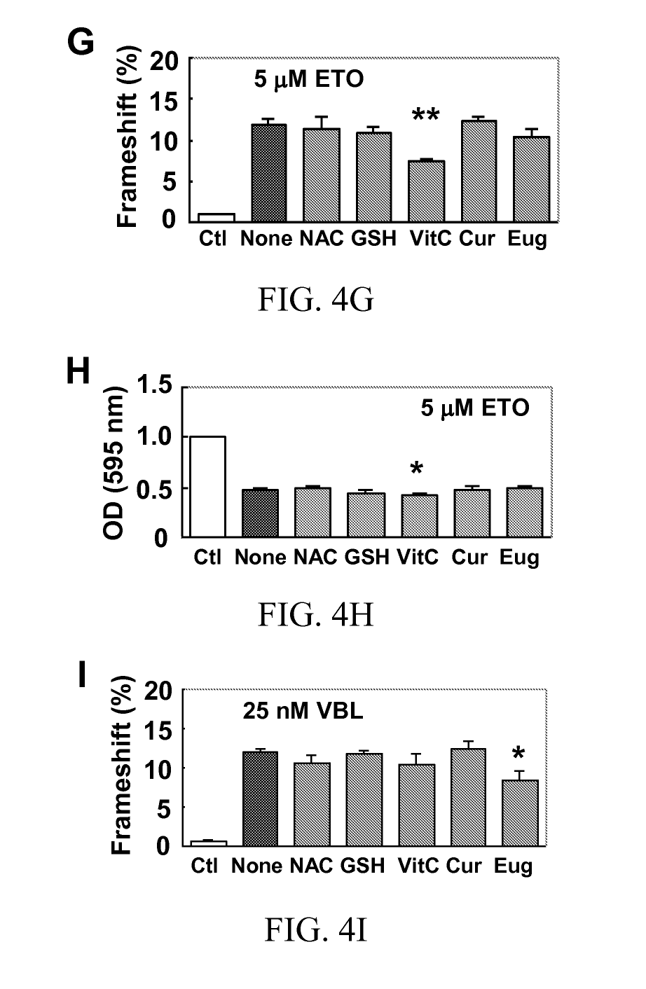

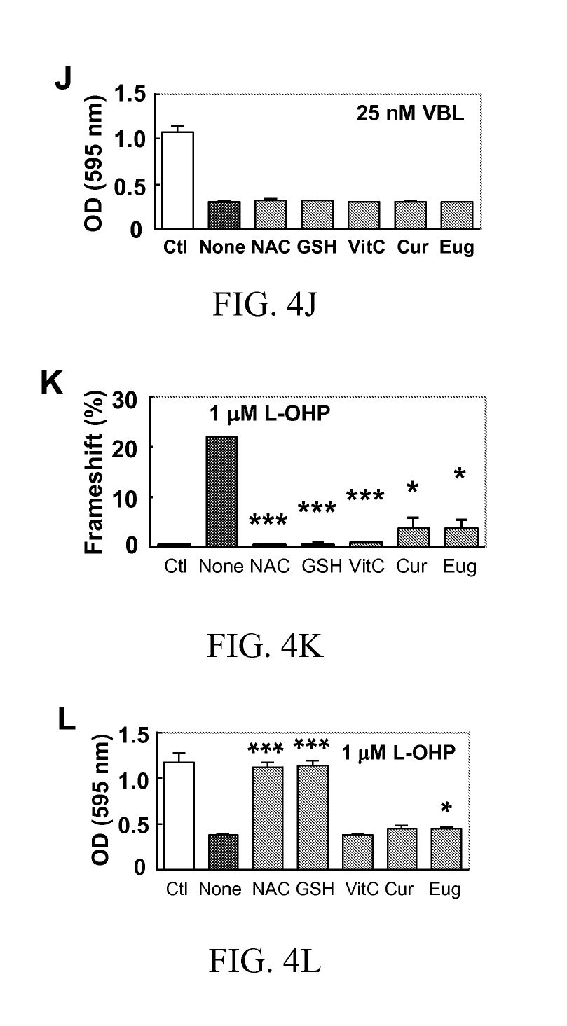

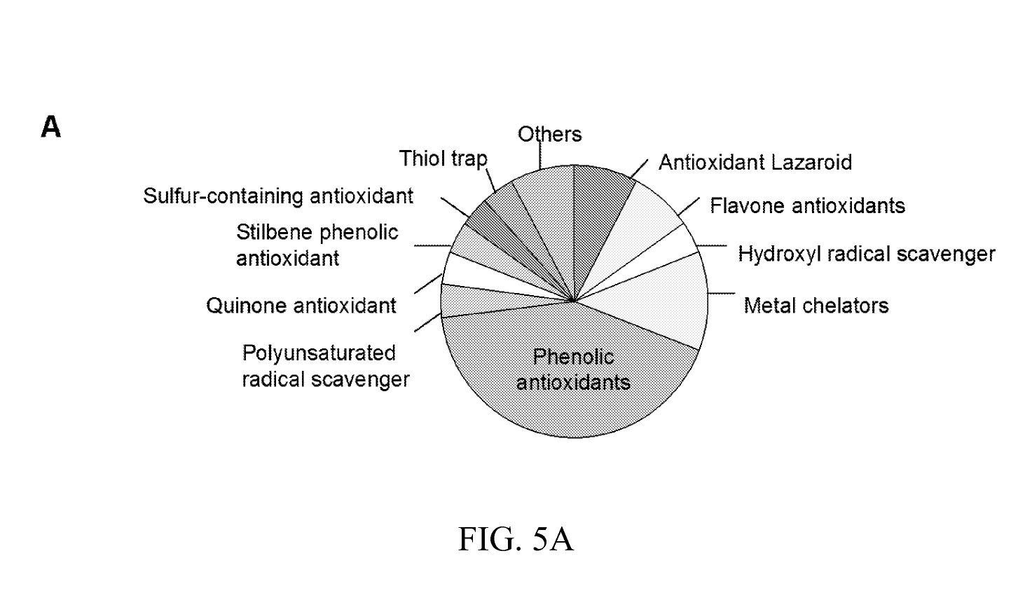

After a 3-day recovery from a 3-day treatment with 10 μM 5-FU, in the presence or absence of an antioxidant, 150 μM eugenol was the only antioxidant that suppressed 44% of drug-induced MSI without affecting 5-FU-mediated cytotoxicity (FIG. 4A and FIG. 4B).

All tested antioxidants individually suppressed MSI induced by 50 μM CCNU, but exerted different effects on the drug cytotoxicity (FIG. 4C and FIG. 4D). Notably, 2.5 mM NAC and 5 mM GSH suppressed 56-66% MSI induced by CCNU, while adversely decreasing CCNU-mediated cytotoxicity by approximately 25% (FIG. 4C and FIG. 4D). Vitamin C, at 250 μM, decreased CCNU-induced MSI by ˜24% without significantly affecting drug cytotoxicity. On the other hand, 1.5 μM curcumin or 150 μM eugenol suppressed CCNU-induced MSI by approximately 55% while positively enhancing CCNU-mediated cytotoxicity by 11-14%.

Methotrexate-induced MSI was suppressed by co-treatment with NAC, GSH or vitamin C by 34%, 13% or 12% respectively without affecting drug cytotoxicity (FIG. 4E and FIG. 4F). Although 1.5 μM curcumin did not affect methotrexate-induced MSI, it positively enhanced methotrexate-mediated cytotoxicity by approximately 15% (FIG. 4E and FIG. 4F).

Only vitamin C suppressed etoposide-induced MSI, by ˜40%, while enhancing etoposide-mediated cytotoxicity by ˜10% (FIG. 4G and FIG. 4H). Similarly, only eugenol suppressed vinblastine-induced MSI by 40% but had no effects on drug cytotoxicity (FIG. 4I and FIG. 4J).

All tested antioxidants dramatically suppressed MSI induced by 1 μM oxaliplatin (FIG. 4K and FIG. 4L). Notably, 2.5 mM NAC and 5 mM GSH totally abolished oxaliplatin-mediated cytotoxicity 25% (FIG. 4K and FIG. 4L). Also, 150 μM eugenol slightly compromised oxaliplatin-mediated cytotoxicity. Collectively, the antioxidants examined appear to differentially affect MSI and the cytotoxicity mediated by the tested drugs.

2.6 the Primary Screen of a REDOX Library.

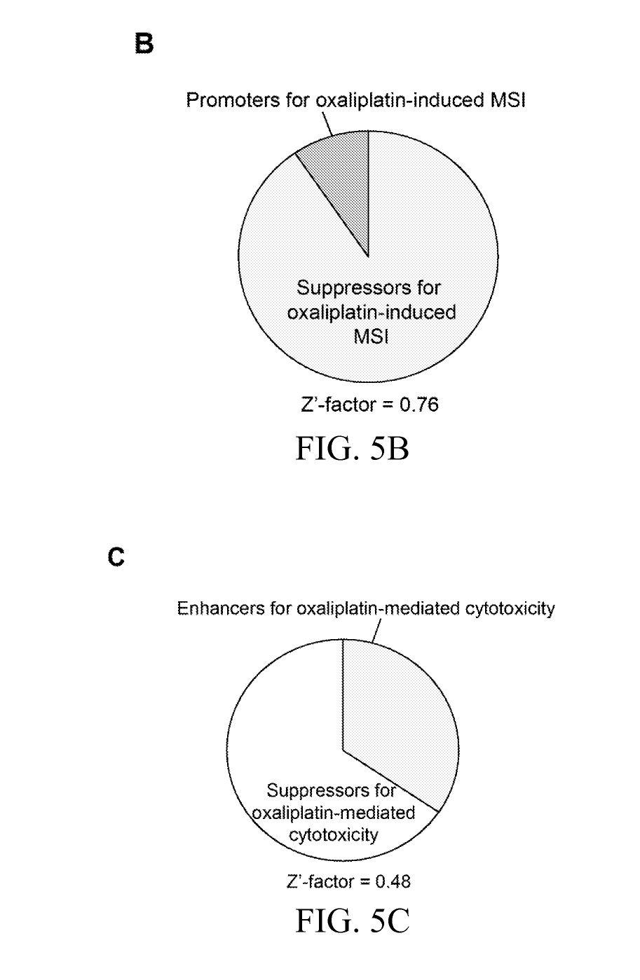

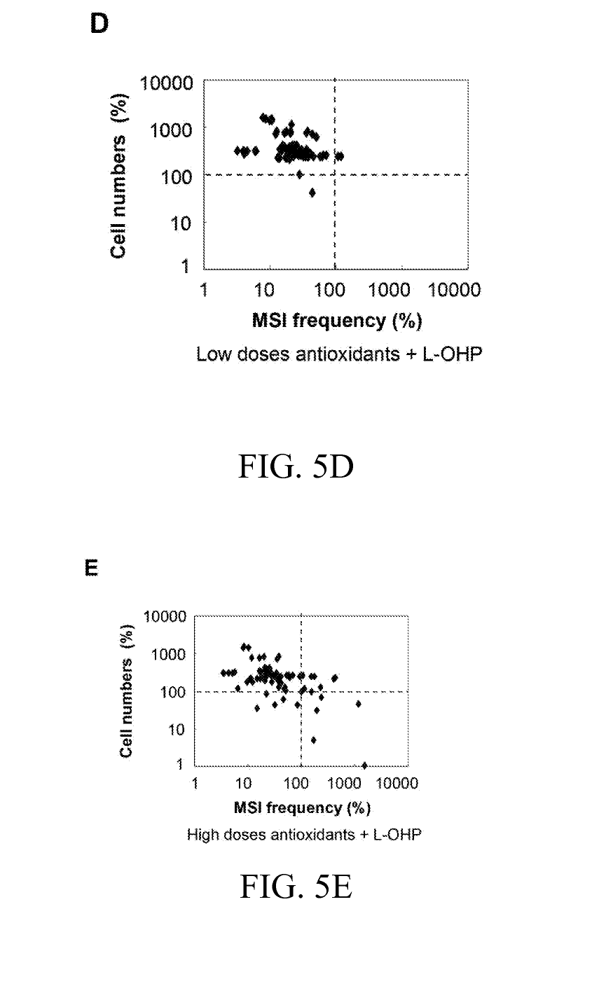

Chemotherapy is generally given in a combination of drugs. For example, the FOLFOX regimen, consisting leucovorin, 5-FU and oxaliplatin, has become a standard regimen for treating patients with high risk stage II and stage III CRC [Andre et al., 2004; Grothey and Sargent, 2005]. Although MMR-deficient cancer cells do not develop resistance to oxaliplatin [Vaisman et al., 1998; Ahmad, 2010], this drug caused MMR deficiency since it displayed strong MSI-inducing and intermediate ROS-generating abilities among tested drugs (FIG. 1A to 1M, FIG. 2A to 2C and FIG. 3). We therefore screened a REDOX library in HCT116-(CA)₁₃ cells to identify antioxidants that can suppress oxaliplatin-induced MSI but do not decrease oxaliplatin-mediated cytotoxicity in HCT116-(CA)₁₃ cells by high-content microscopy. Based on known functions, 42.3% of 84 compounds in the REDOX library are phenolic antioxidants, 11% are metal chelators and 7.7% are flavone antioxidants among others (FIG. 5A). HCT116-(CA)₁₃ cells were manually co-treated in 96-well plates with 1 μM oxaliplatin and each of compounds at its IC₅₀ or 10% of IC₅₀ values, if available in the literature. After a 3-day co-treatment, followed by a 3-day recovery, the cells were fixed, stained, and analyzed for the MSI frequency and cell numbers simultaneously by high-content microscopy. If MSI was analysis only, ˜90% of compounds in the library suppressed oxaliplatin-induced MSI (FIG. 5B). If only cell numbers were analyzed, ˜34% of compounds enhanced oxaliplatin-mediated cytotoxicity (FIG. 5C). To determine the assay quality, the Z′-factor was calculated and was 0.76 for the MSI assay and 0.48 for cell number counting by high-content microscopy. It is considered as an excellent assay, when 1>Z′-factor≧0.5 [Zhang et al., 1999].

We further plotted both MSI frequency and cell numbers together. At a low dose, such as 10% of IC₅₀, the majority of compounds in the library suppressed oxaliplatin-induced MSI while adversely decreasing oxaliplatin-mediated cytotoxicity (FIG. 5D). At a 10-fold higher dose, such as IC₅₀, more compounds suppressed oxaliplatin-induced MSI while enhancing oxaliplatin-mediated cytotoxicity (FIG. 5E). On the other hand, some compounds dramatically enhanced oxaliplatin-mediated cytotoxicity at the expense of increasing oxaliplatin-induced MSI (FIG. 5E). These findings indicate that a clinical value of an antioxidant relies on simultaneously evaluate both drug-induced MSI and drug-mediated cytotoxicity.

2.7 Identification of Antioxidant Candidates from a Secondary Screen

From a list of potential candidates identified in the primary screen, we performed a secondary screen by including additional concentrations.

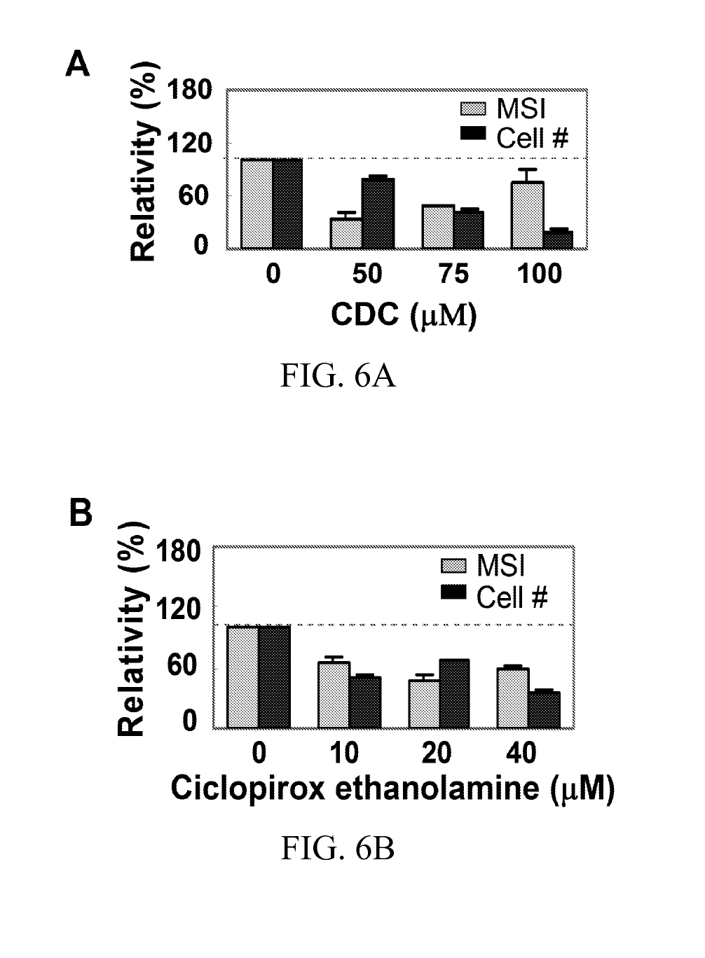

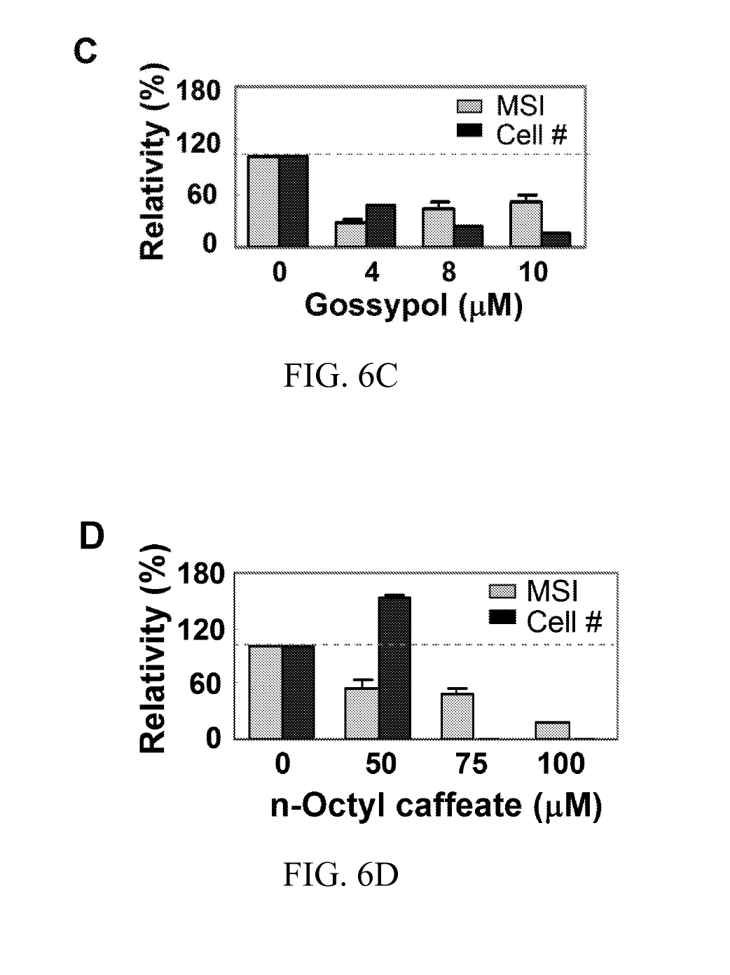

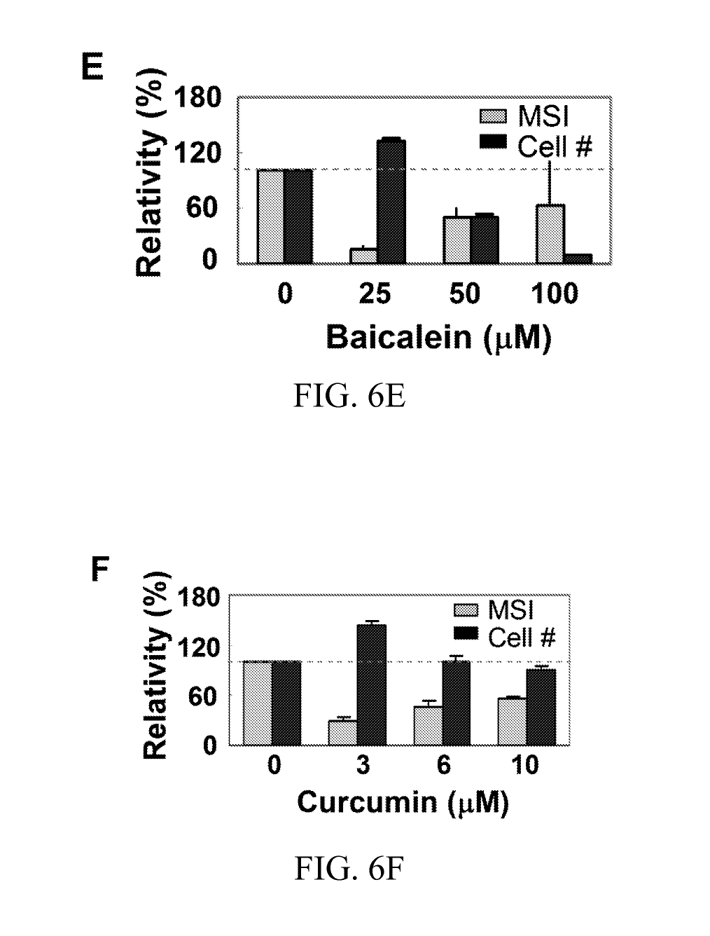

The most promising antioxidants include CDC (FIG. 6A), ciclopirox ethanolamine (FIG. 6B), gossypol (FIG. 6C), n-octyl caffeate (FIG. 6D), baicalein (FIG. 6E) and curcumin (FIG. 6F). Ciclopirox ethanolamine is a hydroxyl radical scavenger, baicalein is a flavone antioxidant and the rest candidates are phenolic antioxidants.

2.8 Effects of Gossypol or Curcumin on Oxaliplatin-Mediated Cytotoxicity of MMR-Deficient and MMR-Deficient Cells

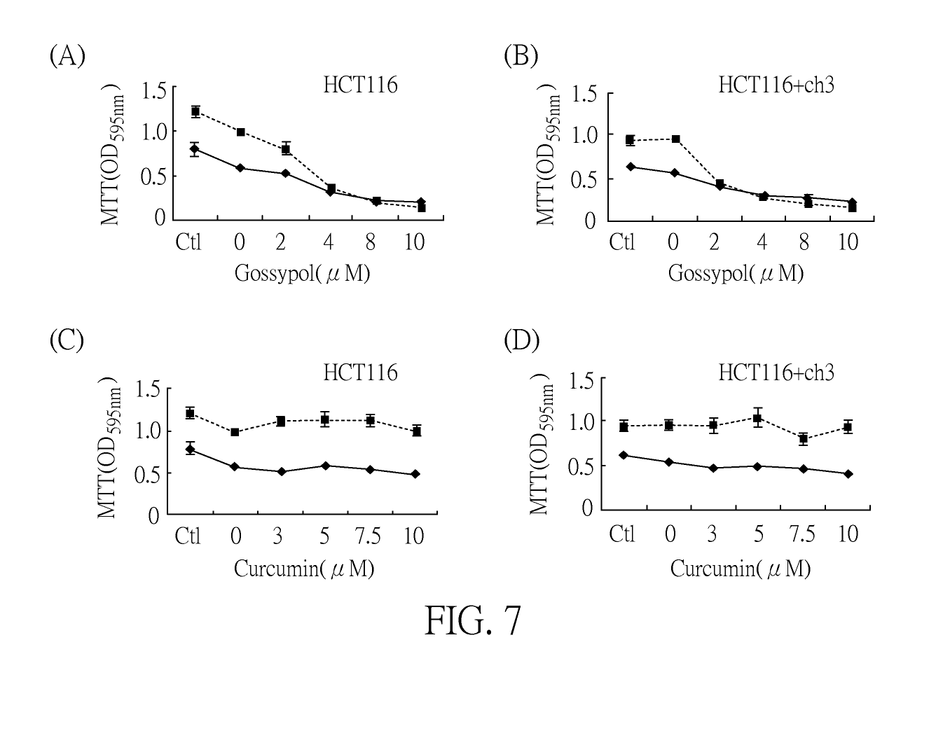

We further investigated whether the MMR status affects the effect of gossypol or curcumin on oxaliplatin-mediated cytotoxicity. MMR-deficient HCT116 cells and isogenic MMR-proficient HCT116+chr3 cells were co-treated with 1 μM oxaliplatin and 2-10 μM gossypol or curcumin. The cytotoxicity was determined by the MTT assay after a 3-day co-treatment with (dotted lines) or without (solid lines) a 3-day recovery. As shown in FIG. 7, Gossypol similarly enhanced oxaliplatin-mediated cytotoxicity in a concentration-dependent manner in both MMR-deficient cells (A) and MMR-deficient cells (B). Three days after recovery from the co-treatment, there was no new proliferation occurred ((A) and (B)). In contrast, curcumin did not show a significant effect on oxaliplatin-mediated cytotoxicity in both MMR-deficient and MMR-deficient cells ((C) and (D)).

Although the present invention has been explained in relation to its preferred embodiment, it is to be understood that many other possible modifications and variations can be made without departing from the spirit and scope of the invention as hereinafter claimed.

What is claimed is:

1. A method for reducing microsatellite instability in chemotherapy, which comprises administering a therapeutically effective amount of an antioxidant to an individual receiving the chemotherapy.

2. The method of claim 1, wherein the antioxidant is effective in suppressing microsatellite instability induced by a chemotherapeutic agent while enhancing an efficacy of the chemotherapeutic agent.

3. The method of claim 1, wherein the chemotherapy is performed by administering a chemotherapeutic agent selected from the group consisting of anti-metabolites, alkylating agents, topoisomerase II poisons, microtubule disruptors, their derivatives, and a combination thereof.

4. The method of claim 3, wherein the chemotherapeutic agent is selected from the group consisting of 5-fluorouracil, lomustine (CCNU), methotrexate, etoposide, vinblastine, oxaliplatin, their derivatives, and a combination thereof.

5. The method of claim 4, wherein the chemotherapeutic agent is oxaliplatin.

6. The method of claim 1, wherein the antioxidant is selected from the group consisting of phenolic antioxidants, flavone antioxidants, hydroxyl radical scavengers, their derivatives, and a combination thereof.

7. The method of claim 6, wherein the antioxidant is selected from the group consisting of CDC, ciclopirox ethanolamine, gossypol, n-octyl caffeate, baicalein, curcumin, their derivatives, and a combination thereof.

8. The method of claim 7, wherein the antioxidant is gossypol.

9. The method of claim 1, wherein the individual is suffered from colorectal cancer.

10. The method of claim 1, wherein the antioxidant is effective in preventing occurrence of secondary cancer in the individual receiving the chemotherapy.

11. The method of claim 1, wherein the antioxidant is effective in inhibiting drug resistance in the individual receiving the chemotherapy.

12. A method of screening compounds useful in reducing microsatellite instability (MSI), by employing first and second cell lines, wherein the first cell line is deficient in a component of the DNA mismatch repair (MMR) system and the second cell line is proficient for DNA mismatch repair (MMR) system that harbor a dual-fluorescent MSI reporter, the method comprising:
(a) contacting the first line with at least one candidate antioxidant with a chemotherapeutic agent;
(b) determining the MSI frequency or amount of cell death in the first cell line;
(c) selecting a promising candidate antioxidant which suppresses drug-induced MSI or enhances drug's cytotoxicity in the first cell line;
(d) determining the MSI frequency or amount of cell death in the second cell line when contacting the promising candidate antioxidant with a chemotherapeutic agent; and
(e) selecting a promising candidate antioxidant which suppresses drug-induced MSI or enhancing drug's cytotoxicity in the second cell line.

13. The method of claim 12, wherein the first and second cells lines are isogenically matched.

14. The method of claim 12, wherein the first and second cells lines are cancer cell lines.


SEQUENCE LISTING (10 sequences)

SEQ ID NO: 1 - DNA, 20 bp, Artificial Sequence
  synthetic sequence
tcgcctccaagaatgtaagt

SEQ ID NO: 2 - DNA, 21 bp, Artificial Sequence
  synthetic sequence
tctgcattttaactatgcctc

SEQ ID NO: 3 - DNA, 21 bp, Artificial Sequence
  artificial sequence
tgactacttttgacttcagcc

SEQ ID NO: 4 - DNA, 22 bp, Artificial Sequence
  synthetic sequence
aaccattcaacatttttaaccc

SEQ ID NO: 5 - DNA, 20 bp, Artificial Sequence
  synthetic sequence
ggaagaatcaaatagacaat

SEQ ID NO: 6 - DNA, 24 bp, Artificial Sequence
  synthetic sequence
gctggccatatatatatttaaacc

SEQ ID NO: 7 - DNA, 20 bp, Artificial Sequence
  synthetic sequence
aaacaggatgcctgccttta

SEQ ID NO: 8 - DNA, 20 bp, Artificial Sequence
  synthetic sequence
ggactttccacctatgggac

SEQ ID NO: 9 - DNA, 21 bp, Artificial Sequence
  synthetic sequence
actcactctagtgataaatcg

SEQ ID NO: 10 - DNA, 25 bp, Artificial Sequence
  synthetic sequence
agcagataagacagtattactagtt